(19) United States
(12) Patent Application Publication
(10) Pub. No.: US 2024/0050607 A1
(43) Pub. Date: Feb. 15, 2024

(54) A LIGHTING AND DISINFECTION DEVICE

(71) Applicant: Energy Research Lab Ltd, London (GB)

(72) Inventor: Lee Harrison, London (GB)

(73) Assignee: Energy Research Lab Ltd, London (GB)

(21) Appl. No.: 18/258,019

(22) PCT Filed: Dec. 20, 2021
(86) PCT No.: PCT/GB2021/053378

§ 371 Date: Jun. 16, 2023

(30) Foreign Application Priority Data
Dec. 18, 2020 (GB) 2020149.7

(51) Int. Cl. A61L 2/10; A61L 2/24; A61L 9/20; H05B 45/335; H05B 45/305

(52) U.S. Cl. CPC A61L 2/10; A61L 2/24; A61L 9/20; H05B 45/335; H05B 45/305

(57) ABSTRACT

The disclosure provides disinfection devices and lighting devices. For example, there is provided a lighting device configured to be driven by an HFAC power supply, the lighting device comprising a light, a first circuit comprising a first secondary coil electrically connected to the light and configured to be coupled to an HFAC power supply, a second circuit electrically connected to the light and comprising a secondary coil configured to be coupled to an HFAC power supply, a HFAC to DC converter, a DC to AC driver, a clock pulse generator and a transformer.

The present disclosure relates to methods and apparatus, and more particularly to methods and apparatus for disinfection by light modulated with very high frequency signals, examples relate to a lighting device oscillating at very high frequencies.

A high number of hospital patients contract healthcare associated infections while they are in hospital that were not present in the patients at the time of their admission. These infections can be caused by multidrug resistant organisms, for example. Healthcare-associated infections sicken millions of people worldwide every year and C difficile is the most commonly reported pathogen. Enhanced disinfection not only benefits the next patient who enters a high-risk room but also indirectly benefits other hospitalised patients. Patients with short-term hospitalisation sometimes develop healthcare associated infections—infections contracted in healthcare facilities. Currently, the most prominent way to combat this in hospitals is by decontaminating the patient wards and rooms by conducting a deep and thorough physical or manual clean and disinfection using chemical disinfectants. In recent years, the development of portable ultraviolet (UV) light systems has been seen which have been proposed to supplement the manual cleaning and disinfecting of the hospital areas. UV light has proven to be effective in decreasing the presence incidents of many infectious organisms such as Ebola and MRSA. There is a need for enhanced room disinfection methods, as standard manual methods have proven largely ineffective at eliminating certain pathogens that can survive on hospital surfaces and in suspension for extended periods and therefore put hospital patients and staff at risk of infection. Applying the same techniques to the domestic home environment provides control methods for effectively reducing the incidence for example of the flu and the common cold.

MRSA as an example has been proven to be recirculated among patients, the air, and the inanimate environments, accelerated by movement in rooms. Airborne MRSA may play a role in MRSA colonisation in the nasal cavity or in respiratory tract MRSA infections. Airborne infections of many types are spread through the air in droplets associated with, for example, coughs or sneezing of infected subjects. UV light has been shown to be effective in controlling areas to reduce infection rates by reducing or killing bacteria and viruses.

Traditional UVC lamp systems have been used in a number of applications. One example is the use of air conditioning systems as a means of combatting so-called “sick building” syndrome and sterilizing associated plant to kill diseases such as Legionella's that breed in water in the system. They are then inadvertently spread around in water vapour.

In addition there are the larger automated UVC room disinfection devices, effectively “robots” that move around rooms autonomously dosing visible surfaces with UVC light. For medical equipment and personal effects, there are a wide number of UV “cabinet” solutions on the market. These bathe objects placed in them in UVC light to disinfect them. Due to the high power required, these solutions have tended to use traditional lamp technologies. While these solutions are extremely effective, there are a range of applications for which traditional UV lamps are unsuitable. Due to their size, fragility, operating temperature, and power supply requirements, it is impractical to mount traditional UV lamps in handheld devices or to embed them directly into medical equipment itself.

UV light is fundamentally divided into four main types and three of them have potential health disadvantages when not correctly used in populated areas;
UW 400-450 nm; No health risk
UVA 315 nm-400 nm; Cataracts of Lens. Skin Cancer, Retinal Burns
UVB 280 nm-315 nm; Corneal Injury, Cataracts of Lens Photo keratitis, Erythema, Skin Cancer
UVC 100 nm-280 nm; Corneal Injury, Photo keratitis, Erythema, Skin Cancer

In addition to UVA, UVB and UVC is UW. UVV is classed as the UV visible transition range between 400-450 nm. UV light with a wavelength of less than 400 nm (UVA, UVB and UVC) damages unprotected human eyes due to the human eye's inability to detect UV. Our normal protection of blinking or pupil contraction in bright light is ineffective in the UV wavelength, therefore to look at a UV light is the same as staring at a bright light without blinking. Due to these disadvantages, some prior art products exist whereby localised areas can be decontaminated only when areas are vacant, but these use either expensive high energy UV sources that must be carefully placed at specific positions within a room, or incorporated in small handheld disinfecting products.

To overcome the potential issues with UV light, some prior art exists whereby light sources in the visible violet region (UVV) 400-450 nm range are used to disinfect areas, a method of disinfection without the damage potential to unprotected eyes and skin caused by UVA, B or C. One of the drawbacks of any light germicidal treatment by means of light is due to the fact that bacteria exist in clusters and dose—response of spores within clusters is affected by the attenuation of UV fluence as it propagates through spores within the cluster. In 2014, Sempeles suggested that narrow-spectrum UV light could be used to kill bacteria and reduce infections without damaging human tissue. The very narrow spectrum of UV light that could be utilised with such capabilities was around 207 nm (Sempeles, S. J. C/in. Eng. 2014. vol 39(1). pp 2-3). UV light around this wavelength is strongly absorbed by proteins, but cannot be absorbed by the nucleus of human cells. However, due to the small size of bacteria cells, the UV light can reach their DNA.

### SUMMARY

Aspects and examples of the invention are set out in the claims.

An aspect of the disclosure provides a disinfection device for disinfecting a surface of an article or a volume of light transmissive fluid, such as a gas or a liquid.

One such disinfection device comprises a UV light source, such as an LED, arranged to be powered by a first alternating current having a first frequency, and further powered by second alternating current superimposed on the first alternating current. The second alternating current has a higher frequency than the first alternating current. This may cause the intensity of the light emitted by the light source to be modulated (e.g., to strobe) at a higher frequency than the frequency of the first alternating current. The light source may comprise a source of UV light.

The disinfection device may be configured to be powered by inductive coupling with the bus of an HFAC power supply system.

The first alternating current may be provided by first power supply circuitry connected to the light source. The first power supply circuitry may comprise a coil for inductive coupling with the bus of an HFAC power supply system. The first power supply circuitry may be arranged so that the first alternating current has the same frequency as the signal carried by the bus of the HFAC power supply system.

The second alternating current may be provided by second power supply circuitry connected to the light source and connected to be powered by inductive coupling with the same bus. The second power supply circuitry may comprise an output which is connected to the light source but which is otherwise DC isolated. For example the connection between the second power supply circuitry and the light source may be provided by a transformer.

The second power supply circuitry may comprise a frequency converter so that the second alternating current has a higher frequency than the first alternating current.

The light source may comprise an LED.

An aspect of the disclosure provides a disinfection device comprising any one or more of the lighting devices described herein, wherein the light source comprises a source of UV light, such as a UV light emitting diode.

An aspect of the disclosure relates to the use of such a lighting device to disinfect a surface of an article by the application to the surface of UV light produced by the light source.

Embodiments of the disclosure provide lighting devices for use with an HFAC power supply of an HFAC power distribution system. Such HFAC systems may comprise a power bus carrying HFAC for powering a plurality of inductively powered devices placed along the power bus.

Some embodiments of the disclosure provide lighting systems and disinfection systems comprising an HFAC power supply system and any one or more of the lighting devices described herein inductively coupled to the power bus of the HFAC power supply system.

The inventors have realised that by combining UV light with High Frequency AC, it is possible to create point of use applications that have been unviable with traditional technology in the drive towards virus and bacteria control, but also to provide disinfection control in many areas and circumstances that are more effective than previously possible.

The inventors have realised that by combining High Frequency AC driven lighting with light sources between 100-450 nm, three very important actions take place. The variable high frequency and current driving the light source results in lighting that to the human eye appears stable, but in fact it is oscillating at a frequency of between 20-850 kHz. This oscillation is undetectable to the human eye but appears stroboscopic to bacterial cells.
Firstly, the HFAC oscillating light source fundamentally damages the DNA of pathogen cells, reducing their ability to replicate.
Secondly, the HFAC light output targets the virus particles' or bacterial spores' resonant frequency, now not only reducing their ability to multiply, but also to effectively ‘shake’ them apart, causing destruction by vibrating apart both the single particles or spores whilst also beginning to vibrate loose cluster packs of cells, exposing any shielded cells such that they too can be targeted again by the HFAC driven light source, effectively removing their shields provided by the cluster cells. This method of driving the light source enables all light sources to become more effective in damaging the DNA of dangerous airborne infectious viruses, with significant ability to reduce the incidence of less dangerous viruses such as the common cold and norovirus for example. All virus, bacterial or other infectious particles and spores have their own resonant frequency whereby damage is irreversible with no effect on humans due to the inability of the HFAC driven light to penetrate the human body.
The inventors have further realised as the capsid of a pathogen, particularly a virus is like the shell of a turtle, by using HFAC emitted at varying frequencies via light in the 100-450 nm range the shell can be compromised as the effect is similar to mechanical vibrations, thence the virus can be inactivated. Laser pulses tuned to the right ultrasonic frequency can kill certain viruses, for example low frequency ultrasound on MRSA at 35 kHz reduced a 1 million colony forming units (CFU) of MRSA to 6 CFU after just 30 seconds. The results suggest that 35-kHz Low Frequency Ultrasound reduces CFU of bacteria, punctures and fractures cell walls, and alters colonial characteristics of MRSA, including resistance to the oral form of methicillin. Further research has proved that Far UVC light, a narrow band of UVC light at 200 nm wavelength cannot penetrate the dead layer of skin or the outer layer of the eye, is safely absorbed by proteins and other molecules in the skin and is unable to reach the nucleus of normal human cells, thereby safe for use in populated areas. However, bacterial cells are 10-25 times smaller than human cells and, therefore, still susceptible to the far-UVC's damaging rays. The inventors have realised that by driving UV lighting from variable 100-450 nm wavelengths with HFAC LED, drive current at variable frequency, 20-850 kHz and current, 10 mA to 3200 mA, the resulting HFAC UV light ‘sweeps the area’, destroying any bacterial sources within its reach, combining resonance with UV light for ultimate effect. Viruses and bacteria can become resistant to drug therapy, they cannot become resistant to mechanical vibration causing their destruction.

As different pathogens are resonant at different frequencies it is desirable to provide a way to target pathogens at specific frequencies. Additionally, different pathogens may be susceptible to different wavelengths.

According to the invention there is provided a lighting device configured to be driven by an HFAC power supply, the lighting device comprising a light, a first circuit comprising a first secondary coil electrically connected to the light and configured to be coupled to an HFAC power supply, a second circuit electrically connected to the light and comprising a secondary coil configured to be coupled to an HFAC power supply, a HFAC to DC driver, a DC to AC driver, a clock pulse generator; and a transformer.

Although an HFAC power supply system supplies power at a high frequency, the frequency may not be that of the resonant frequency of a specific microbe or pathogen. By controlling the frequency of the high frequency AC power source driving the light, the oscillation frequency can be more carefully controlled. Furthermore different lights can oscillate at different frequencies in order to target different microbes or pathogens.

The LED operates at a single wavelength but different LEDs may use different wavelengths. For example there may be a plurality of LEDs all oscillating at the same frequency but operating at different wavelengths.

The lighting device can be used to disinfect an area, a surface, an airspace or a portion of fluid. By exposing pathogens to a light source at a resonant frequency pathogens such as viruses and bacteria are therefore destroyed and eliminated. In this way a surface, area, airspace or portion of fluid may be decontaminated of pathogens.

The light is preferably an LED and preferably a UV LED.

The clock pulse generator may generate a pulse at a frequency of between 1 kHz and 1 MHz. There may additionally be a pulse timer module configured to switch the clock pulse generator on and off at predetermined intervals. The pulse timer module switches the clock pulse generator on and off at for up to 100 ms every 1 s or 5 s.

The lighting device may further comprise a second light electrically connected only to the second circuit. Thus it is driven only by the frequency of the second circuit and not powered by the current in the first circuit.

The lighting device may comprise a third light and a third electrical circuit electrically connected to the second light and comprising a secondary coil configured to be coupled to an HFAC power supply, an HFAC to DC driver, a DC to AC driver, a clock pulse generator and a transformer. Thus there may be additional secondary circuits such that there are a plurality of different lights all being drive at different frequencies. Furthermore the second light may operate at a different wavelength from the first light

The AC to DC converter may be configured to adjust the output voltage to between 1 and 50V. The DC to AC driver may comprise a switching device and may comprise a half bridge or full bridge. The HFAC to DC converter may comprise synchronous rectification.

The clock pulse generator comprises at least one of: discrete logic gates, a micro controller, a field programmable gate array or a digital signal processor.

According to the invention there is provided a method of lighting comprising generating an electrical signal first frequency and superimposing a second frequency onto the first frequency to driving a light with the combined signal. The secondary frequency may be superimposed for an interval, followed by a period where it is not superimposed.

### FIGURES

FIG. 1 depicts a lighting device according to the invention;

FIG. 2 depicts an alternative light according to the invention; and

FIG. 3 depicts an alternative light according to the invention.

### DESCRIPTION OF A HIGH FREQUENCY AC SYSTEM

A high frequency AC system (HFAC) generates an alternating frequency at frequencies above 1 kHz or 10 kHz. This can be used for wireless powering of devices and lights. There is generally a power bus to which devices are inductively coupled. The invention relates to a lighting device to be used in conjunction with an HFAC system.

### Lighting Device

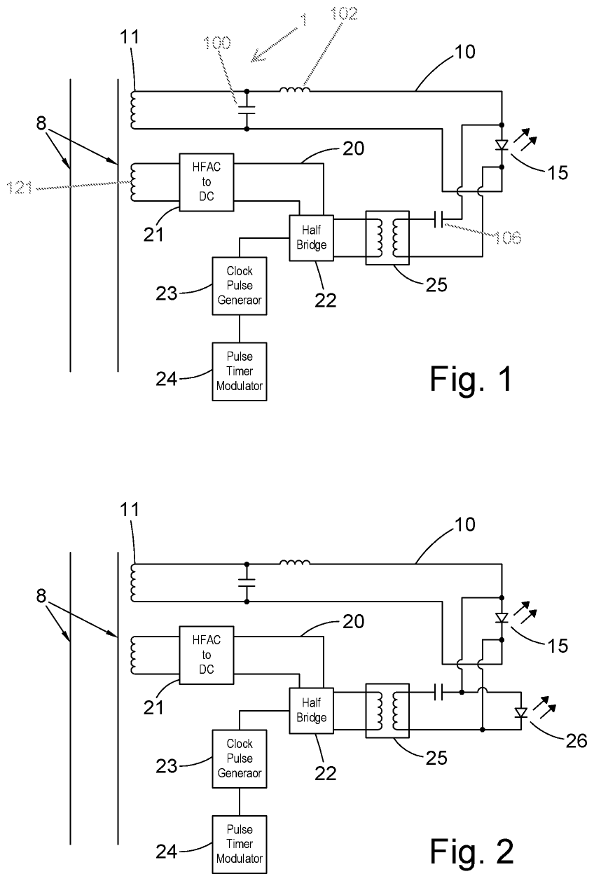

FIG. 1 depicts an apparatus comprising a lighting device 1 according to the invention. The apparatus shown in FIG. 1 also comprises a power bus 8 carrying HFAC electrical power.

The device comprises a first circuit 10 which is inductively coupled to the power bus 8 by a coil 11. The device 1 may comprise a capacitor 100 connected in parallel across the coil 11. A first end of the coil 11 may be connected by an inductor 102 to a first connection of a light emitting diode, LED, 15. A second end of the coil 11 is connected to a second connection of the LED 15. Thus, HFAC alternating current induced in the coil 11 is supplied to the series connection of the inductor 102 and the LED 15, in parallel with the capacitor 100.

The alternating current induced in the coil 11 is dependent on the number of turns. For example, the current induced in the coil 11 may be 0.375 A, induced by a current of 1.5 A in the power bus. The first circuit powers a UV LED 15 and has a plurality of resistors and a capacitor. The AC frequency of the first circuit is determined by the frequency of the bus from which it draws a current. For example, if the power bus oscillates at a frequency of 20 kHz the first circuit will have an AC current of 20 kHz and the light on the lighting circuit will be powered at 20 kHz.

The lighting device 1 also comprises a secondary circuit 20 with a secondary coil 121. The secondary circuit 20 is configured to obtain an HFAC power supply from the HFAC bus 8 and to provide a second alternating current based on that HFAC power supply of a higher frequency than the HFAC power supply. The secondary electrical circuit 20 is connected to provide this second alternating current to the LED 15.

The first circuit 10 and the secondary circuit 20 are both connected to the LED so that the alternating current provided by the first circuit 10 and the alternating current provided by the secondary circuit 20 combine additively (are superimposed on each other) to provide a combined power supply to the LED 15.

The secondary circuit 20 illustrated in FIG. 1 comprises an HFAC to DC converter 21, which may comprise a synchronous (e.g., active) rectifier for providing a rectified voltage output. The HFAC to DC converter 21 is connected to the secondary coil 21 for receiving HFAC power. The output from the HFAC to DC converter is a voltage which may be in the range 1-50V. This DC voltage is input into a half bridge circuit 22 (or alternatively a full bridge circuit) which converts the signal back to an AC signal, for example at a frequency higher than the frequency of the HFAC supply derived from the bus 8 by the secondary coil 121. The frequency of the AC signal generated by the half bridge circuit is controlled by a clock pulse generator 23. The clock pulse may be operating at a frequency of 200 kHz to generate an oscillating frequency output by the half bridge of 100 kHz. The signal is output to a transformer 25 which is electrically connected to the UV LED 15. A primary coil of the transformer 25 is connected to the output of the half bridge. A first end of the secondary coil of the transformer 25 is connected by a capacitor 106 to the first connection of the light emitting diode, LED, 15 (and so via the inductor 102 also to the first end of the coil 11 of the first circuit 20. A second end of the secondary coil of the transformer 25 is connected to the second connection of the light emitting diode, LED, 15. This provides the second alternating current to the LED 15.

It can also be seen from FIG. 1 that the voltage on the secondary coil of the transformer 25 is referenced to the first coil 20 and DC isolated from the half bridge 22 and its DC power supply 21. The transformer 25 and capacitor 106 may form a resonant tank comprising an LC circuit. Other configurations of capacitor(s) and/or additional inductor(s) may also be used, for example to provide an LLC circuit, and LCL circuit, an LCL-T circuit or any other LC circuit which forms a resonant tank.

The secondary circuit 20 may be operable to control the second alternating current so that it is switched on and off for selected intervals. These intervals may be periodic or intermittent. For example, the secondary circuit 20 illustrated in FIG. 1 comprises an optional pulse timer module 24. This switches the clock pulse generator off and on for a predetermined period. It might switch the clock pulse generator on for a few hundred microseconds in every few seconds. For example it might switch the clock pulse generator on for 100 ms every 5 seconds. Any appropriate duty cycle can be selected. Outside the cony periods the clock pulse generator will not be operational and will therefore not generate a clock signal to control the half bridge. There will not therefore be an alternating current input to the transformer 25 and so no additional high frequency will be transmitted to the UV LED. Thus outside the time when the clock pulse generator is on (i.e. when the clock pulse generator is off) the UV LED will not oscillate at the additional high frequency. This is because powering LEDs at very high frequencies such as 300 kHz for prolonged periods could damage the LED, Oscillating the LED at very high frequencies alone is therefore limited to short durations. Other methods of modulating the second alternating current applied to the LED may also be used.

The UV LED oscillates at a frequency controlled by the first circuit 10. However, there are additional oscillations at a frequency controlled by the second circuit. Thus the UV LED may operate at a frequency of 20 kHz (generated by the first circuit) but have additional, smaller oscillations at 300 kHz (generated by the secondary circuit). The smaller oscillations are smaller and superimposed onto the larger oscillations at 20 kHz.

Driving separate channels of high frequency AC, for example, 300 KHz at 1 to 200 mA can be used alongside channels driving LEDs at for example 20 KHz to provide a solution in which many frequencies can be covered simultaneously by superimposing a higher frequency on a lower frequency for a predetermined time. For example a 300 KHz signal can be superimposed on a 20 KHz output for 10 ms every 1 second for example.

It can therefore be seen that the embodiment of FIG. 1 offers one way to provide an LED which is arranged to powered by a first alternating current at a first power supply frequency, and further powered by a second alternating current superimposed on the first alternating current. The second alternating current has a higher frequency than the first so that the LED strobes with the first frequency and with the second higher frequency, higher than the frequency of the HFAC power supply.

Although the use of an LED as a light source is described above other light sources such as mercury vapour, black lights, curing lamps, germicidal lamps, halogen lights, high-intensity discharge lamps, fluorescent and incandescent sources, and some types of lasers may alternatively be used.

The number of cons on the primary coil 11 and the secondary coil 21 will affect the current and voltages at which the first and second circuit operate. The first circuit may operate at a higher current and voltage than the second circuit, or alternatively the second circuit may operate at a higher current and voltage than the first circuit.

The wavelength, or colour, of the emitted light is controlled by the chemical composition of the light itself. The frequency with which the light switches on and off, or the variation in intensity of the light, is controlled by the frequency at which the current oscillates.

FIG. 2 is identical to FIG. 1 except it includes an additional LED 26. The additional LED is connected in parallel with the LED 15 of the arrangement shown in FIG. 1. This circuit may be configured so that, rather than being connected in parallel with the first LED 15, the additional LED 26 is powered only by the secondary circuit and is therefore switched on and off with the pulse timer module, at a frequency controlled by the clock pulse generator. The additional LED will oscillate at very high frequencies (controlled by the clock pulse generator) but will only operate for very short periods, controlled by the pulse timer module.

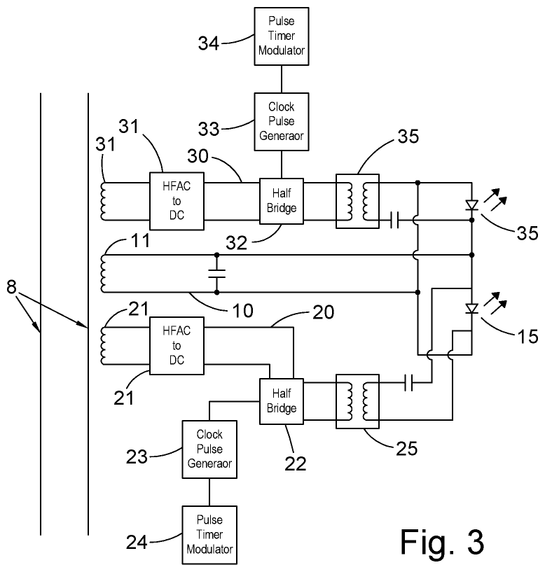

FIG. 3 depicts an alternative light according to the invention in which there is a third circuit 30 and a second LED 35 electrically connected to the third circuit. The third circuit is similar to the second circuit 20 and comprises an HFAC to DC converter 31, a half bridge circuit 32, a clock pulse generator 33, a pulse timer module 34, and a transformer 35. The clock pulse generator of the third circuit operates independently of the clock pulse generator of the second circuit and therefore the second and third circuits can operate at different AC frequencies. Thus different secondary circuits power different LEDs and can oscillate at different frequencies.

All the LEDs 15, 26, 35 described above may also be controlled by switches to switch the LEDs off when not needed.

Although FIG. 3 depicts an arrangement with second and third circuits there could be any number of additional (secondary) circuits and corresponding lights/LEDs. Different LEDs may operate at different wavelengths. A lighting device may therefore include a plurality of LEDs which are individually controllable. For example, a user could select light operating at a particular wavelength e.g. 400 nm and oscillating at a particular resonant frequency of a target pathogen. Furthermore, a plurality of the lights within a single lighting device could operate simultaneously with a first light operating at a first wavelength and oscillating at a first frequency and a second light operating at a second wavelength and oscillating at a second frequency.

The intervals the pulse timer module operates at may be dependent on the switching frequency of the clock pulse generator. For example, if a higher switching frequency is used there may be shorter periods where the clock pulse generator is on, or longer intervals between the on periods. Conversely when a lower switching frequency is used (to generate a lower AC frequency) longer on-periods may be used, or shorter periods between the on=periods.

Each lighting device may draw a power of up to 100 W. As discussed above, several lights, or LEDs may operate simultaneously such that the combination of lights operate at a plurality of frequencies and a plurality of wavelengths to target specific pathogens.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A disinfection device comprising:
a light source arranged to be powered by a first power supply circuitry providing a first alternating current having a first frequency and by a second power supply circuitry providing a second alternating current superimposed on the first alternating current,
wherein the second alternating current has a higher frequency than the first alternating current to cause the intensity of the light emitted by the light source to be modulated at a higher frequency than the frequency of the first alternating current.

2. The device of claim 1 wherein the light source comprises light emitting diode, LED, for example an ultra violet, UV, LED.

3. The device of claim 1 or 2 wherein the second power supply circuitry is configured to switch the second alternating current on and off to provide a series of pulses of alternating current.

4. The device of claim 3 wherein each pulse comprises the second alternating current being switched on for between 1 ms and 1 second, for example 100 ms.

5. The device of claim 4 wherein the series of pulses are provided in a repeating pattern which repeats with a period of between 10 ms and 10 seconds.

6. The device of any of claims 3 to 5 wherein the series of pulses have a duty cycle of between 2% and 10%.

7. The device of any preceding claim wherein an output connection of the first power supply circuitry is connected to an output connection of the second power supply circuitry, and the output connection of the second power supply circuitry is DC isolated from the rest of the second power supply circuitry.

8. The device of any preceding claim wherein the second frequency is between 1 kHz and 1 MHz, for example 100 kHz.

9. The device of any preceding claim wherein the first power supply circuitry comprises a first coil configured to provide inductive coupling with the bus of an HFAC power supply system, and the second power supply circuitry comprises a second coil configured to provide inductive coupling with the bus, and further comprises an HFAC to DC converter connected to the second coil.

10. The device of claim 9 comprising a DC to AC driver connected to the HFAC to DC converter for receiving a DC power supply.

11. The device of claim 9 or 10 comprising a clock pulse generator arranged for providing clock pulses to the DC to AC driver.

12. The device of claim 7 or any preceding claim as dependent thereon wherein the second power supply circuitry comprises a transformer connected between the output of the DC to AC driver and the light source to provide said DC isolation.

13. A lighting device configured to be driven by an HFAC power supply, the lighting device comprising:
a light;
a first circuit comprising a first secondary coil electrically connected to the light and configured to be coupled to an HFAC power supply;
a second circuit electrically connected to the light and comprising:
a secondary coil configured to be coupled to an HFAC power supply;
a HFAC to DC converter;
a DC to AC driver;
a clock pulse generator; and
a transformer.

14. A lighting device according to claim 13 wherein the light is an LED.

15. A lighting device according to claim 14 wherein the LED is a UV LED.

16. A lighting device according to any of claims 13 to 15 wherein the clock pulse generator generates a pulse at a frequency of between 1 kHz and 1 MHz.

17. A lighting device according to any one of claims 12 to 16 further comprising a pulse timer module configured to switch the clock pulse generator on and off at predetermined intervals.

18. A lighting device according to claim 17 wherein the pulse timer module switches the clock pulse generator on for a time of 1 ms to 1000 ms every 10 ms to 10 seconds.

19. A lighting device according to any one of claims 13 to 18 further comprising a second light electrically connected only to the second circuit.

20. A lighting device according to any one of claims 13 to 19 and further comprising a third light and a third electrical circuit electrically connected to the second light and comprising:
a secondary coil configured to be coupled to an HFAC power supply;
an HFAC to DC converter;
a DC to AC driver;
a clock pulse generator; and
a transformer.

21. A lighting device according to claim 20 wherein the third light operates at a different wavelength from the first light.

22. A lighting device according to any one of the preceding claims wherein the HFAC to DC converter is configured to adjust the output voltage to between 1 and 50V.

23. A lighting device according to any one of the preceding claims wherein the DC to AC driver comprises a half bridge or full bridge.

24. A lighting device according to any one of the preceding claims wherein the HFAC to DC converter comprises synchronous rectification.

25. A lighting device according to any one of the preceding claims wherein the clock pulse generator comprises at least one of: discrete logic gates, a micro controller, a field programmable gate array or a digital signal processor.

26. A lighting device according to any one of the preceding claims further comprising a switch for each light.

27. A method of lighting comprising:
generating a first electrical signal at a first frequency;
superimposing a second electrical signal having a second frequency onto the first frequency to provide a combined electrical signal, and driving a light with the combined signal.

28. The method of lighting according to claim 27 wherein the second frequency is superimposed for an interval, followed by a period where it is not superimposed.

29. Use of the device of any of claims 1 to 26 to disinfect an article, wherein the light source comprises a UV light source.

30. A disinfection device comprising the lighting device of any of claims 13 to 26 wherein the light source of the lighting device comprises a UV light source.
